(19) United States
(12) Patent Application Publication
(10) Pub. No.: US 2017/0319142 A1
(43) Pub. Date: Nov. 9, 2017

(54) Optical Measurements while in Motion

(71) Applicant: John William Peterman, JR., Madison, WI (US)

(72) Inventor: John William Peterman, JR., Madison, WI (US)

(21) Appl. No.: 15/145,984

(22) Filed: May 4, 2016

(51) Int. Cl. A61B 5/00; A61B 5/024; G01L 1/24

(52) U.S. Cl. CPC A61B 5/721; G01L 1/24; A61B 5/02427; A61B 5/681

(57) ABSTRACT

This invention provides improved optical measurements while in motion by collecting a force signal that measures how hard the optical device is pressing on the body. The force signal is used by a noise-canceling algorithm to reduce motion artifacts from the desired optical signal.

### CROSS-REFERENCE TO RELATED APPLICATIONS

This application claims the benefit of PPA Ser. No. 62/156,661 filed May 4, 2015 by the present inventor

### FEDERALLY SPONSORED RESEARCH

Not Applicable

### SEQUENCE LISTING OR PROGRAM

Not Applicable

### BACKGROUND OF THE INVENTION

### Field of Invention

This invention relates to optical measurement methods used in medical instrumentation.

### Background of the Invention

Optical detection is a method used to monitor a number of factors in humans and animals. It consists of shining a light source on a portion of the body, detecting the transmitted or reflected light then analyzing the resulting data to determine levels of the factor of interest or monitor changes which can determine pulse. Measurements while the body is in motion such as in medical transport or during activities such as exercise are of growing interest. Problems with the technique are encountered when the motion causes changes in the optical path changes in the detected signal often to the point of obscuring the desired signal. Minor changes in the optical path can cause large effects in the optical signal. Detection of the pulse during exercise is a common application. Separation of the pulse signal from the exercise signal can be very difficult particularly if the exercise is rhythmic such as running and if the pace is in the same range as typical pulse. Current state of the art uses accelerometers to detect the motion. While the signal provided by the accelerometer does indicate the motion of the body, it may or may not indicate any change in the optical signal so is not of much value as a correction signal. If a signal representing the change in the optical signal due to motion is available it can be mathematically reduced from the optical signal through the use of an adaptive algorithm.

There are a number of optical detection methods currently used for monitoring. Pulse Oximetry is an optical method used to detect the pulse and the oxygenation level of the blood. It is done by shining infrared and red light either through the skin or reflected from the skin and detecting and calculating the ratio between the two. Carbon Monoxide is detected the same manor but using a different wavelength where the Carbon Monoxide preferentially absorbs light. Glucose level detection is another use as are others.

In addition to the transmittance and reflectance measurement methods Photoacoustic and Raman spectroscopy are optical methods that can also be used and can benefit from motion artifact reduction. Photoacoustic uses a pulsed source which when it is absorbed by a compound, heats it slightly causing an expansion which causes a sound which can be detected Raman is similar to reflection only it detects emitted light, which has been sifted slightly from the source wavelength. The optical elements are comprised of sources and detectors. With the different techniques the source can vary from one to several LEDs, or a laser, a lens or fiber optic which is providing the source light to the body. The detectors can be one or several optical detectors, a lens or fiber optics or a microphone for Photoacoustic.

To reduce the motion artifacts from the optical signal, a noise cancellation algorithm such as an adaptive filter is used. FIG. 2 shows the input to the algorithm is the signal from the optical detector that includes the signal of interest plus noise such as from the motion and other sources. The other input is a signal which represents of the undesired change or noise, which the algorithm will optimally remove from the detector signal leaving the signal of interest. Noise cancellation and Adaptive filters are described in numerous papers such as Widrow et al. Adaptive Noise Cancelling: Principles and Applications. Proceedings of the IEEE, Vol 63, No. 12, December 1975.

We have found that measurement of the force between the optical sensors and the body produces a signal, which correlates to the variation in the optical path, which contributes to artifacts or noise.

Measurement of force can be performed in a number of ways. One method is through the use of thin film sensors. Thin film force sensors are composed of a number of layers or flexible material usually plastic, which surround an active layer which response to changes in force by changing its resistance. Thin film sensors because of their small size and flexibility are useful for this device, but other measurement methods such as inductive, capacitive and magnetic can be used.

Location of the force sensor can varied in a number ways as long as it is physically connected, if not physically touching, the optical elements and the body so it is able to monitor the change in force of the optical elements on the body being testing. One example is implemented by using a thin film force sensor located under the optical source and detector utilizing a sensor that transmits the wavelength of light for the analysis or simply has holes where needed for light passage. Another method is to mount the source and detector on the force sensor. In a system such as watch or armband, the sensor could be mounted on the band opposite of the optical sensor or monitoring the band tension. Further enhancements are to incorporate the force sensor into the same chip as the source leds and detector.

The problem with an accelerometer used by a number of systems is that the signal does not necessarily correlate with the undesired changes in the optical signal because it is only the change in the optical path or body part monitored with relationship to the sensor that is of interested, i.e. if both are accelerated together the optical response will not change but the accelerometer does.

An electronics module located near the optical sensor and the force sensor collects data from each simultaneously or interleaved so they are monitoring at the same time. The data can be fully processed by this module to reduce noise and calculate and display resulting values such as pulse or Oxygen levels. This module can be implemented as a circuit comprised of individual components, or a microprocessor or a combination of the two. The data may be partially processed locally or not at all and simply transmitted to another system such as a smart phone, via bluetooth, or WIFI where further processing is done and results displayed.

Other embodiments include moving the sensor to other locations that can provide a good noise signal caused by motion this could be on the other side of the body or the force sensor could be integrated in the band and be monitoring the tension of the band.

In addition to noise cancellation, it is anticipated the force signal can be used as a start signal to collect data only when the sensor is in contact with the skin. The force signal can be further analyzed to detect pulse directly, or to indicate exercise parameters such as pace. Other variations include the application of several force sensors such as once associated with the optical source and another with the optical detector in cases where the two a some distance apart.

### BRIEF SUMMARY OF THE INVENTION

The invention improves optical measurements methods used on humans and animals that may be in motion by using an integrated force sensor to provide a noise signal, which is used mathematically to reduce the artifacts in the signal, caused by subject motion.

### DRAWINGS—FIGURES

FIG. 1 side, cutaway view of the invention as a watch

FIG. 2 block description of a noise-canceling algorithm

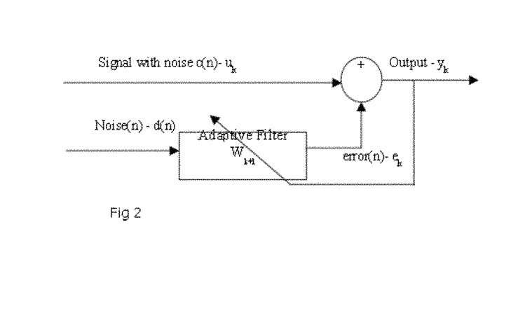

### DRAWINGS—REFERENCE NUMBERS

1 Cutaway of a wristband to hold the device to the body
2 Electronics box
3 Thin film force sensor
4 Optical source
5 Optical detector
6 Blood

### DETAILED DESCRIPTION OF THE INVENTION

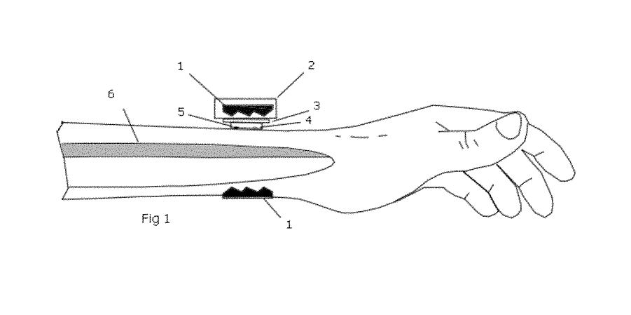

The present invention uses a force sensor to detect changes in the force between the optical sensors and the skin being measured where we have found there is a direct correlation between the optical signal artifacts and the force signal. FIG. 1 show a representation of one possible arrangement of the components of the system.

In this configuration, which can be applied to the body like a watch or an armband, the optical sensor 5 and light source/s 4 are mounted on a thin film force sensor 3 and the entire package is held to the body by a band 1 (shown cut away). The Source 5 and Sensor 4 monitor the blood in the body 6. An electronics module which includes the source and the sensor like the MAX30100 from Maxim Integrated San Jose, Calif. can be used and mounted on a thin-film sensor such as the FSR400 from Interlink Electronics, Inc, Westlake Village. Data is collected on a microprocessor such as an ARM 1pc1768 from NXP Semiconductors Netherlands B.V. Eindhoven The Netherlands.

I claim:

1) a device to reduce motion effects on optical body measurements, comprising:
(a) an optical source,
(b) an optical detector,
(c) a force sensor physically connected to the optical elements and the body such that produces a force signal representing the contact between the optical elements and the body being monitored,
(d) an electronics module to collect data from the force sensor and the optical detector and to apply an adaptive algorithm using the force signal as a noise input to produce an improved optical signal.

2) a device to reduce motion effects on optical body measurements, comprising:
(a) an optical source,
(b) an optical detector,
(c) a force sensor physically connected to the optical elements and the body such that produces a force signal representing the contact between the optical elements and the body being monitored,
(d) a circuit and microprocessor to collect data from the force sensor and the optical detector and send the data to another device which applies an adaptive algorithm using the force signal as a noise input to produce an improved optical signal.

3) a method of reducing artifacts in optical measurements made while in motion which uses a signal from a force sensor measuring the contact force of the optical elements to the body being monitored to be used as a noise signal in a noise cancellation algorithm.
